(19) United States
(12) Patent Application Publication
(10) Pub. No.: US 2002/0040018 A1
(43) Pub. Date: Apr. 4, 2002

(54) Subtype selective melatonergics

(76) Inventor: Robert M. Jones, Salt Lake City, UT (US)

Correspondence Address: ROTHWELL, FIGG, ERNST & MANBECK, P.C., 555 13TH STREET, N.W., SUITE 701, EAST TOWER, WASHINGTON, DC, 20004, US

(21) Appl. No.: 09/886,609

(22) Filed: Jun. 22, 2001

Related U.S. Application Data
(60) Provisional application No. 60/264,695, filed on Jan. 30, 2001.
(60) Provisional application No. 60/304,189, filed on Jun. 23, 2000.

(51) Int. Cl. A61K 31/55; A61K 31/4745; A61K 31/407

(52) U.S. Cl. 514/214.01; 514/284; 514/410

(57) ABSTRACT

The invention relates to the use of MT₂ selective melatonergics as anticonvulsant agents and as analgesic agents. More specifically, the invention relates to the use of 6H-isoindolo[2,1-a]indoles or 5,6-dihydroindolo[2,1-a]isoquinolines as described herein which have melatonin agonist activity and which are selective for the MT₂ receptor as anticonvulsant agents or analgesic agents. The invention further relates to the use of 5,6-dihydroindolo[2,1-a]isoquinolines and 6,7-dihydro-5H-benzo[c]azepino[2,1-a]indoles as described herein which have melatonin antagonist activity and which are selective for the MT₂ receptor as pharmacological tools for delineation of physiological responses governed by MT₂ receptor activation either by melatonin or selective agonists disclosed herein and for treatment of disorders associated with overproduction of melatonin such as seasonal affective disorder (SAD) and shift work syndrome. Such melatonin antagonists are also useful for treating Parkinson's Disease.

### CROSS-REFERENCE TO RELATED APPLICATIONS

The present application is related to U.S. provisional patent application No. 60/______ filed on Jun. 23, 2000 (attorney docket number 2314-183) and to U.S. provisional patent application No. 60/264,695 filed on Jan. 30, 2001, each incorporated by reference herein. The present application further claims priority to each of these applications.

### BACKGROUND OF THE INVENTION

The invention relates to the use of MT₂ selective melatonergics as anticonvulsant agents and as analgesic agents. More specifically, the invention relates to the use of 6H-isoindolo[2,1-a]indoles or 5,6-dihydroindolo[2,1-a]isoquinolines as described herein having melatonin agonist activity and which are selective for the MT₂ receptor as anticonvulsant agents or analgesic agents. The invention further relates to the use of 5,6-dihydroindolo[2,1-a]isoquinolines and 6,7-dihydro-5H-benzo[c]azepino[2,1-a]indoles as described herein having have melatonin antagonist activity and which are selective for the MT₂ receptor as pharmacological tools for delineation of physiological responses governed by MT₂ receptor activation either by melatonin or selective agonists disclosed herein and for treatment of disorders associated with overproduction of melatonin such as seasonal affective disorder (SAD) and shift work syndrome. Such melatonin antagonists are also useful for treating Parkinson's Disease.

The publications and other materials used herein to illuminate the background of the invention, and in particular, cases to provide additional details respecting the practice, are incorporated by reference, and for convenience are referenced in the following text by author and date and are listed alphabetically by author in the appended bibliography.

Melatonin (N-acetyl-5-methoxytryptamine) is a hormone which is synthesized and secreted primarily by the pineal gland. Melatonin levels show a cyclical, circadian pattern with highest levels occurring during the dark period of a circadian light-dark cycle. Melatonin is involved in the transduction of photoperiodic information and appears to modulate a variety of neural and endocrine functions in vertebrates, including the regulation of reproduction, body weight and metabolism in photoperiodic mammals, the control of circadian rhythms and the modulation of retinal physiology. Physiological and pharmacological doses of melatonin elicit profound chronobiotic and hypnotic effects which undoubtedly suggest a therapeutic axis for treatment of insomnia and circadian rhythm sleep disorders. Furthermore recent data has illustrated an immunomodulatory effect for the hormone as well as an ability to inhibit cell proliferation in certain cancers.

Recent evidence demonstrates that melatonin exerts its biological effects through specific G-protein coupled receptors (GPCRs). Use of the biologically active, radiolabelled agonist [¹²⁵I]-2-iodomelatonin has led to the identification of high affinity melatonin receptors in the CNS of a variety of species. The sequences of two cloned human melatonin receptors have been reported [Reppert et al., 1995; Reppert et al., 1994). In mammalian brain, autoradiographic studies have localized the distribution of melatonin receptors to a few specific structures. Although there are significant differences in melatonin receptor distribution even between closely related species, in general the highest binding site density occurs in discreet nuclei of the hypothalamus. In humans, specific [¹²⁵I]-2-iodomelatonin binding within the hypothalamus is completely localized to the suprachiasmatic nucleus, strongly suggesting the melatonin receptors are located within the human biological clock.

Exogenous melatonin administration has been found to synchronize circadian rhythms in rats (Cassone et al., 1986). In humans, administration of melatonin has been used to treat jet-lag related sleep disturbances, considered to be caused by desynchronization of circadian rhythms (Arendt et al., 1986). Further, the use of a single dose of melatonin to induce sleep in humans has been claimed by Wurtman in International Patent Application WO 94/07487.

Epilepsy is a recurrent paroxysmal disorder of cerebral function characterized by sudden brief attacks of altered consciousness, motor activity, sensory phenomena or inappropriate behavior caused by abnormal excessive discharge of cerebral neurons. Convulsive seizures, the most common form of attacks, begin with loss of consciousness and motor control, and tonic or clonic jerking of all extremities but any recurrent seizure pattern may be termed epilepsy. The term primary or idiopathic epilepsy denotes those cases where no cause for the seizures can be identified. Secondary or symptomatic epilepsy designates the disorder when it is associated with such factors as trauma, neoplasm, infection, developmental abnormalities, cerebrovascular disease, or various metabolic conditions. Epileptic seizures are classified as partial seizures (focal, local seizures) or generalized seizures (convulsive or nonconvulsive). Classes of partial seizures include simple partial seizures, complex partial seizures and partial seizures secondarily generalized. Classes of generalized seizures include absence seizures, atypical absence seizures, myoclonic seizures, clonic seizures, tonic seizures, tonic-clonic seizures (grand mal) and atonic seizures. Therapeutics having anticonvulsant properties are used in the treatment of seizures. Most therapeutics used to abolish or attenuate seizures act at least through effects that reduce the spread of excitation from seizure foci and prevent detonation and disruption of function of normal aggregates of neurons. Traditional anticonvulsants that have been utilized include phenytoin, phenobarbital, primidone, carbamazepine, ethosuximide, clonazepam and valproate. Several novel and chemically diverse anticonvulsant medications recently have been approved for marketing, including lamotrigine, ferlbamate, gabapentin and topiramate. For further details of seizures and their therapy, see Rall & Schleifer (1985) and The Merck Manual (1992).

Pain, and particularly, persistent pain, is a complex phenomenon involving many interacting components. Chronic or intractable pain, which may result from degenerative conditions or debilitating diseases, is currently treated with a variety of analgesic compounds, often opioid compounds such as morphine. Likewise, neuropathic pain, typically a chronic condition attributable to injury or partial transection of a peripheral nerve, is also conventionally treated with opioid compounds such as morphine. Conventional therapies for pain produce analgesia—a loss of sensitivity to pain without the loss of consciousness. Opioid compounds have been used widely to produce analgesia, including plant-derived opioids such as morphine, and endogenous opioids such as met- and leu-enkephalins, as well as β-endorphin. Opioid compounds, while effective in producing analgesia for many types of pain, may induce tolerance in some patients. When a patient becomes tolerant, increasing doses of the opioid are required to produce the desired analgesic effect. In addition, these compounds frequently result in a physical dependence in patients, and may have side effects at high doses.

It is desired to develop new anticonvulsant agents for the treatment of epilepsy and to develop new analgesic agents for the treament of acute and chronic pain. It is also desired to develop new agents for identifying conditions associated with MT₂ receptor activation.

### SUMMARY OF THE INVENTION

The invention relates to the use of MT₂ selective melatonergics as anticonvulsant agents and as analgesic agents. More specifically, the invention relates to the use of 6H-isoindolo[2,1-a]indoles or 5,6-dihydroindolo[2,1-a]isoquinolines as described herein which have melatonin agonist activity and which are selective for the MT₂ receptor as anticonvulsant agents or analgesic agents. The invention further relates to the use of 5,6-dihydroindolo[2,1-a]isoquinolines and 6,7-dihydro-5H-benzo[c]azepino[2,1-a]indoles as described herein which have melatonin antagonist activity and which are selective for the MT₂ receptor as pharmacological tools for delineation of physiological responses governed by MT₂ receptor activation either by melatonin or selective agonists disclosed herein and for treatment of disorders associated with overproduction of melatonin such as seasonal affective disorder (SAD) and shift work syndrome. Such melatonin antagonists are also useful for treating Parkinson's Disease.

The methods of this invention are useful in the treatment of pain (whether acute or chronic), including chronic pain, and neuropathic pain, without undesirable side effects, and in the prevention or treatment of convulsions, including epilepsy.

### BRIEF DESCRIPTION OF THE FIGURES

FIGS. 1A and 1B show the effect of melatonin in phase one (1A) and phase two (1B) of the formalin model of persistent pain.

FIGS. 2A and 2B show the effect of CGX-031133 in phase one (2A) and phase two (2B) of the formalin model of persistent pain.

FIGS. 3A and 3B show the effect of CGX-031139 in phase one (3A) and phase two (3B) of the formalin model of persistent pain.

FIGS. 4A and 4B show the effect of CGX-MTAG in phase one (4A) and phase two (4B) of the formalin model of persistent pain.

FIGS. 5A-5D show the effect of melatonin (5A), CGX-031139 (5B), CGX-031133 (5C) and CGX-MTAG (5D) on motor impairment in the accelerating rotorod test.

### DETAILED DESCRIPTION OF THE INVENTION

The present invention is directed to the use of compounds of Formula I as either (a) anticonvulsant agents and analgesic agents or (b) agents for delineating conditions associated with MT₂ activation or overproduction of melatonin. Compounds useful as anticonvulsant agents and analgesic agents demonstrate selectivity for the MT₂ receptor and have melatonin agonistic activity. Compounds useful as agents for delineating conditions associated with MT₂ activation and overproduction of melatonin demonstrate selectivity for the MT₂ receptor and have melatonin antagonistic activity. These latter compounds are useful for treating seasonal affective disorder (SAD), shift work syndrome and Parkinson's Disease. Compounds of Formula I are prepared as described in Faust et al. (2000).

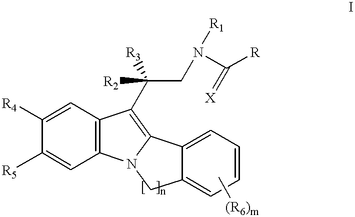

wherein

R is H, a C₁₋₆ alkyl, CF₃, C₂F₅, C₃₋₆ cycloalkyl, —(CH₂)ₚ—C₃₋₆ cycloalkyl, C₂₋₆ alkenyl, C₂₋₆ alkynyl or heterocycle

R₁ is H or C₁₋₃ alkyl; or

R and R₁ together with the atoms to which they are attached form a heterocyclic ring of 5-7 atoms;

R₂ and R₃ are independently H or C₁₋₃ alkyl; or

R₂ and R₃ together with the atom to which they are attached form a C₃₋₆ cycloalkyl;

R₄ is a H, OR⁷ or SR⁷;

R₅ is H, C₁₋₅ alkyl, phenyl, halogen (preferably F or Cl); or

when R₅ is a C₁₋₅ alkyl, then R₅ may also be linked to R₄ by an O or an S;

R₆ is H, halogen (preferably F or Cl), C₁₋₄ alkyl, C₁₋₄ alkoxy, C₁₋₄ thioalkyl, phenyl or heterocycle;

R₇ is H, C₁₋₆ alkyl or —(CH₂)ₚ—C₃₋₆ cycloalkyl;

X is O, S or NH;

m is 0, 1 or 2

n is 0, 1, 2 or 3; and

p is 0, 1, 2, 3 or 4.

The alkyl groups may be straight or branched chain and be unsubstituted or substituted with a halogen atom (e.g., fluorine, chlorine, bromine, iodine), a nitro group, a cyano group, a hydroxy group, an amino group, a carboxy group, a C₁₋₃ alkoxy, a halogenated C₁₋₃ alkyl group, a mono- or di-C₁₋₃ alkylamino group, a C₁₋₃ alkylcarbonyl group, a C₁₋₃ alkoxycarbonyl group, a carbamoyl group, and a mono- or di-C₁₋₃ alkylcarbamoyl group. The substituted alkyl groups may have 1 to 5, preferably 1 to 3 substituents selected from those mentioned above, at any substitutable positions in the group. When the number of the substituents is two or more, each of the substituents may be the same or different.

The term heterocycle represents a stable, optionally substituted or unsubstituted, saturated or unsaturated monocyclic or bicyclic ring, each ring having 5 or 6 atoms and each ring having from one to four heteroatoms that are the same or different and that are selected from the group consisting of sulfur, oxygen and nitrogen. Examples of heterocycles include, but are not limited to, furan, pyrrole, thiophene, pyrrolidine, pyridine, imidazole, oxazole, thiazole, imidazole, isothiazole, pyrazole, pyrazine, pyrimidine, quinoline, isoquinoline, indole, oxadiazole, thiadiazole, triazole, tetrazole, oxatriazole, thiatriazole, benzo[b]thiophene, benzofuran, tetrahydrobenzofuran and indoline.

The agonist or antagonist activity of any given compound falling within Formula I is readily determined by the Xenopus melanophore assay (Faust et al., 2000). As described therein, agonist activity is determined in the absence of added melatonin, and antagonist activity is determined in the presence of melatonin. Generally, compounds in which n is 0 or 1 have melatonin agonist acivity and compounds in which n is 2 or 3 have melatonin antagonist activity. However, some compounds in which n is 2 have melatonin agonist activity and some compounds in which n is 1 have melatonin antagonist activity. See Faust et al. (2000) for a comparison of agonist and antagonist activity of compounds of Formula I.

A potential role for melatonin in the etiology of epilepsy was reported as early as 1982 by Albertson et al. (1981), although beneficial effects were only observed at high doses. Sugden (1983) claimed in a comprehensive study published in 1983 that there was a clear difference in the potency of melatonin as a sedative/hypnotic and as an analgesic and anticonvulsant. This separation of activities supported an earlier contention that low doses of melatonin have specific sleep promoting action. The anticonvulsant effects of melatonin were the subject of a number of conflicting reports from the early 1970's, Sugden's study concluded that the doses required to facilitate an anticonvulsant action (significant effect v PTZ at 200 mg/kg; ED₅₀ v 3-MPA, 115 mg/kg; ED₅₀ v ECS, 159 mg/kg) are similar to those which produce significant motor incoordination at this pre-dose interval and therefore this effect may not be a specific neuropharmacological action but rather an inability of the experimental animal to make an appropriate motor response. A more recent study reported considerably lower doses of chronically administered melatonin (25 μg for 13 weeks), reduced the number and severity of seizures induced by PTZ in rodents. These promising animal studies have prompted at least 2 recent studies in humans. In the first melatonin was shown to be useful as adjunctive therapy in the clinical control of severe infantile myoclonic epilepsy. Furthermore Fauteck et al. (1999) reported that at a single dose of 5-10 mg melatonin exerted a positive effect on the frequency of epileptic attacks in children with sleep disturbances of various etiologies. In vitro experiments suggested that activation of melatonin receptors in the noecortex were the origins of such observations.

The present invention demonstrates that synthetic MT₂ subtype selective melatonergic agonists, such as CGX-031-120, are useful in treating the effects of seizure in the audiogenic mouse model. As described herein, this study attempted to separate the mt₁ associated “sedation” elicited by the neurohormone from the anti-convulsant properties, which had been speculated to be associated with MT₂ activation. The N-butanoyl-2-(2-methoxy-6H-isoindolo-[2,1a]-indol-11-yl)-ethanamine (IIK7) scaffold has recently been disclosed as a selective MT₂ agonist in standard in vitro melanophore assay of agonist efficacy with accompanying 140 fold selectivity for the MT₂ receptor subtype in radioligand binding studies using clones expressed in NIH-3T3 cells (Sugden et al., 1999) and furthermore a broad series of azepino, isoquinoline and isoindolo[2,1-a]indoles melatonergic derivatives have very recently been reported (Faust et al. 2000). The present invention demonstrates that compounds of Formula I as defined herein to have agonistic activity, such as CGX-031-120 (N-propanol-2-(2-methoxy-6H-isoindolo-[2,1a]-indol-11-yl)-ethanamine) are effective anti-convulsant agents. CGX-031-120 displayed an ED₅₀=77 mg/kg and a TD₅₀>800 mg/kg in the audiogenic mouse model of epilepsy. Melatonin, in a parallel study, was almost equipotent with CGX-031-120, with an ED₅₀=83 mg/kg but with an associated TD₅₀=363 mg/kg. Comparison of the protective indices (PI: CGX-031-120>10 and melatonin=4) illustrates that there is a clear separation of the motor toxicity induced by melatonin and that induced by selective MT₂ agonism. This data compares favorably with similar studies performed for some commercial anti-seizure drugs, e.g., Valproate (Depakote®, Abbott Laboratories) and Ethosuximide.

Melatonin has already been clearly implicated in some psychopharmacological effects including the sedative/hypnotic, anticonvulsant and anti-nociceptive activity (Geoffriau, 1998; Sugden, 1983). In particular, the accumulated reports have shown that melatonin indeed has a potent long lasting analgesic or antinociceptive effect (Golombek et al., 1991; Lakin et al., 1981; Shaji and Kulkarni, 1998; Yu et al., 1999a, b). Recent work by Yu et al. (1998) further illustrated that these antinociceptive effects were centrally mediated by the CNS.

Although the mechanism is poorly understood it is generally assumed that melatonin exerts its effects via centrally expressed melatonin receptors. Melatonin receptors are highly expressed in the mammalian hypothalamus (Morgan et al., 1994; Stankov et al., 1991). A very recent study by Yu et al. (2000) illustrated the effects of Luzindole (MT₂ antagonist) and prazosin (MT₃ antagonist) on melatonin induced anti-nociception using the rat hot water tail flick assay. They reported that ip melatonin (30, 60, 120 mg/kg) resulted in a dose-dependent anti-nociceptive effect, which was antagonized by i.c.v Luzindole (50 & 100 μg) but not by prazosin. Taken together these results infer that melatonin induced anti-nociception is mediated via MT₂ receptors that are located in the central nervous system of the rat. As disclosed herein, compounds of Formula I, such as CGX-031-120, represent a MT₂ selective, orally available, high affinity agonist which is demonstrated herein to have analgesic activity in seveal pain models. The MT₂ receptor subtype therefore represents a novel therapeutic target for analgesia and affords novel selective agonists as described herein which are devoid of the side affects associated with traditional opioid regimens. Furthermore, the compounds described herein appear to be devoid of motor toxicity and represent novel adjuncts for pain therapy for co-administration with COX inhibitors, opioids, sodium channel blockers and other classes of analgesics.

Additionally, the present invention also encompasses stereoisomers as well as optical isomers, e.g., mixtures of enantiomers as well as individual enantiomers and diastereomers, which arise as a consequence of structural asymmetry in certain compounds of Formula I. Separation of the individual isomers is accomplished by application of various methods which are well known to practitioners in the art.

Compounds of Formula I having agonist activity are useful in compositions and methods for the anticonvulsive and analgesic uses. The anticonvulsive agents of the invention have advantages over similar agents. They perform significantly better in maximal electroshock (MES) tests than reference compounds, e.g., phenobarbital and valproic acid. In anticonvulsant studies using pentylenetetrazol (PTZ)-induced seizure techniques, these compounds generally improve length of survival or delay initial twitch or seizure responses.

The compounds of Formula I having agonist activity are also of use in the treatment of disorders which arise from a disturbed functioning of systems which are regulated by melatonin. In particular the compounds of Formula I having agonist activity may be used in the treatment of chronobiological disorders, especially in the elderly population, glaucoma, cancer, psychiatric disorders, neurodegenerative diseases or neuroendocrine disorders arising as a result of or influenced by the systems which are regulated by melatonin.

Chronobiological disorders include seasonal affective disorders (SAD), primary and secondary insomnia disorders, primary and secondary hypersomnia disorders, sleep-wake schedule disorders (including advanced phase type, delayed phase type, disorganised type and frequently-changing type) and other dyssomnias, especially those caused by ageing, dementias, blindness, shift work and by rapid time-zone travel, commonly known as jet lag. Cancers which may be treated with a compound of Formula I having agonist activity include solid tumours, e.g. melanomas and breast carcinomas.

Psychiatric disorders which may be related to altered melatonin function or influenced by melatonin and circadian rhythms include mood disorders (including bipolar disorders of all types, major depression, dysthymia and other depressive disorders), psychoactive substance dependence and abuse, anxiety disorders (including panic disorder, agoraphobia, social phobia, simple phobia, obsessive-compulsive disorder, post-traumatic stress disorder and generalised anxiety disorder), schizophrenia, epilepsy and epileptic seizures (including grand mal, petit mat, myoclonic epilepsy and partial seizures), disorders of involuntary movement (including those due to Parkinson's disease, and drug-induced involuntary movements) and dementias (including primary degenerative dementia of the Alzheimer type).

Neurodegenerative diseases which may be related to altered melatonin function or influenced by melatonin and biological rhythms include multiple sclerosis and stroke.

Neuroendocrine disorders which may be related to altered melatonin function or influenced by melatonin and biological rhythms include peptic ulceration, emesis, psoriasis, benign prostatic hyperplasia, hair condition and body weight. Particular neuroendocrine disorders which may be treated include those relating to the regulation of reproductive maturation and function include idiopathic delayed puberty, sudden infant death, premature labour, infertility, antifertility, premenstrual syndrome (including late luteal phase dysphoric disorder) and sexual dysfunction (including sexual desire disorders, male erectile disorder, post-menopausal disorders and orgasm disorders). The compounds may also be used to manipulate breeding cycles, body weight, coat colour and oviposition of susceptible hosts, including birds, insects and mammals.

The compounds of Formula I having agonist activity may also have sedative and analgesic effects, effects on the microcirculation and immunomodulant effects and may be useful for the treatment of hypertension, migraine, cluster headache, fibromyalgia, regulation of appetite and in the treatment of eating disorders such as obesity, anorexia nervosa and bulimia nervosa.

The compounds of Formula I having antagonist activity are useful as pharmacological tools for delineation of physiological resposes governed by MT₂ receptor activation either by melatonin or by the subtype selective agonists disclosed herein. The compounds of Formula I having antagonist activity are further usefuel for treatment of disorders associated with overproduction of melatonin, such as seasonal affective disorder (SAD) and shift work syndrome. Such melatonin antagonists are also useful for treating Parkinson's Disease.

For example, a potential role for melatonin in Parkinson's diseases was reported in 1999 by Willis and Armstrong (1999). The effects of endogenous and exogenous melatonin on experimental models of Parkinson's disease was tested in Sprague-Dawley rats by exposing them to intracerebroventricular implants of slow release melatonin, pinealectomy, or constant light and then injected with central 6-hydroxydopamine (6-OHDA) or i.p. 1-methyl-4-phenyl,1-1,2,3,6-tetrahydropyridine (MPTP). The resulting impairment of motor function and related behavioural impairment were exacerbated by melatonin implantation, while pinealectomy and exposure to constant light significantly reduced the severity of experimental Parkinson's disease. These results are consistent with previous work highlighting the importance of aberrant amine production in neurological disease and demonstrate that treatments that reduce endogenous melatonin bioavailability can ameliorate experimental Parkinson's disease.

Pharmaceutical compositions containing a compound of the present invention as the active ingredient can be prepared according to conventional pharmaceutical compounding techniques. See, for example, Remington's Pharmaceutical Sciences, 18th Ed. (1990, Mack Publishing Co., Easton, Pa.). Typically, an effective amount, e.g., an antagonistic amound for use as an anticonvulsant or analgesic, of the active ingredient will be admixed with a pharmaceutically acceptable carrier. The carrier is acceptable in the sense that it is compatible with the other ingredients of the formulation and is not deleterious to the recipient thereof. The carrier may take a wide variety of forms depending on the form of preparation desired for administration, e.g., intravenous, oral, parenteral or inhalation. The compositions may further contain antioxidizing agents, stabilizing agents, preservatives and the like.

For oral administration, the compounds can be formulated into solid or liquid preparations such as capsules, pills, tablets, lozenges, melts, powders, suspensions or emulsions. In preparing the compositions in oral dosage form, any of the usual pharmaceutical media may be employed, such as, for example, water, glycols, oils, alcohols, flavoring agents, preservatives, coloring agents, suspending agents (e.g. sorbitol syrup, methyl cellulose or hydrogenated edible fats), emulsifying agents (e.g. lecithin or acacia), preservatives (e.g. methyl or propyl-p-hydroxybenzoates or sorbic acid), and the like in the case of oral liquid preparations (such as, for example, suspensions, elixirs and solutions); or carriers such as starches, sugars, diluents, granulating agents, lubricants (e.g. magnesium stearate, talc or silica), binders (e.g. pregelatinised maize starch, polyvinylpyrrolidone or hydroxypropyl methylcellulose), disintegrating agents (e.g. potato starch or sodium starch glycollate), fillers (e.g. lactose, microcrystalline cellulose or calcium phosphate), wetting agents (e.g. sodium lauryl sulphate) and the like in the case of oral solid preparations (such as, for example, powders, capsules and tablets). Because of their ease in administration, tablets and capsules represent the most advantageous oral dosage unit form, in which case solid pharmaceutical carriers are obviously employed. If desired, tablets may be sugar-coated or enteric-coated by standard techniques. The active agent can be encapsulated to make it stable to passage through the gastrointestinal tract while at the same time allowing for passage across the blood brain barrier. See for example, WO 96/11698. Suitable agents for stable passage may include phospholipids or lecithin derivatives described in the literature, as well as liposomes, microparticles (including microspheres and macrospheres). Alternatively, liquid preparations may be presented as a dry product for constitution with water or other suitable vehicle before use.

For topical administration in the mouth, the compositions may take the form of buccal or sub-lingual tablets, drops or lozenges formulated in conventional manner.

For topical administration to the epidermis the compounds may be formulated as creams, gels, ointments or lotions or as a transdermal patch. Such compositions may for example be formulated with an aqueous or oily base with the addition of suitable thickening, gelling, emulsifying, stabilising, dispersing, suspending and/or colouring agents.

For parenteral administration, the compound may be dissolved in a pharmaceutical carrier and administered as either a solution or a suspension. Illustrative of suitable carriers are water, saline, dextrose solutions, fructose solutions, ethanol, or oils of animal, vegetative or synthetic origin. The carrier may also contain other ingredients, for example, preservatives, suspending agents, solubilizing agents, buffers and the like. When the compounds are being administered intrathecally, they may also be dissolved in cerebrospinal fluid. Parenteral administration may be by injection, conveniently intravenous, intramuscular or subcutaneous injection, for example by bolus injection or continuous intravenous infusion. Formulations for injection may be presented in unit dosage form e.g. in ampoules or in multi-dose containers, with an added preservative. Alternatively, the active ingredient may be in powder form for constitution with a suitable vehicle, e.g. sterile pyrogen-free water, before use.

The compounds of the invention may also be formulated in rectal compositions such as suppositories or retention enemas, e.g. containing conventional suppository bases such as cocoa butter or other glyceride.

Pessaries for vaginal administration may be formulated in a similar manner.

For intranasal administration the compounds of the invention may be used, for example, as a liquid spray, as a powder or in the form of drops.

For administration by inhalation the compounds according to the invention are conveniently delivered in the form of an aerosol spray presentation from pressurised packs or a nebuliser, with the use of a suitable propellant, e.g. 1,1,1,2-trifluoroethane (HFA 134A) and 1,1,1,2,3,3,3-hepta-fluoropropane (HFA 227), carbon dioxide or other suitable gas. In the case of a pressurised aerosol the dosage unit may be determined by providing a valve to deliver a metered amount. Capsules and cartridges of e.g. gelatin for use in an inhaler or insufflator may be formulated containing a powder mix of a compound of the invention and a suitable powder base such as lactose or starch.

Administration of the active agent according to this invention may be achieved using any suitable delivery means, including:

(a) pump (see, e.g., Luer & Hatton (1993), Zimm et al. (1984) and Ettinger et al. (1978));

(b), microencapsulation (see, e.g., U.S. Pat. Nos. 4,352,883; 4,353,888; and 5,084,350);

(c) continuous release polymer implants (see, e.g., U.S. Pat. No. 4,883,666);

(d) macroencapsulation (see, e.g., U.S. Pat. Nos. 5,284,761, 5,158,881, 4,976,859 and 4,968,733 and published PCT patent applications WO92/19195, WO 95/05452);

(e) naked or unencapsulated cell grafts to the CNS (see, e.g., U.S. Pat. Nos. 5,082,670 and 5,618,531);

(f) injection, either intraparentally, subcutaneously, intravenously, intraarterially, intramuscularly, or to other suitable site;

(g) oral administration, in capsule, liquid, tablet, pill, or prolonged release formulation; or

(h) via a patch.

In one embodiment of this invention, an active agent is delivered directly into the CNS, preferably to the brain ventricles, brain parenchyma, the intrathecal space or other suitable CNS location, most preferably intrathecally.

Alternatively, targeting therapies may be used to deliver the active agent more specifically to certain types of cells, by the use of targeting systems such as antibodies or cell-specific ligands. Targeting may be desirable for a variety of reasons, e.g. if the agent is unacceptably toxic, if it would otherwise require too high a dosage, or if it would not otherwise be able to enter target cells.

The active agent is preferably administered in an therapeutically effective amount. The actual amount administered, and the rate and time-course of administration, will depend on the nature and severity of the condition being treated. Prescription of treatment, e.g. decisions on dosage, timing, etc., is within the responsibility of general practitioners or specialists, and typically takes account of the disorder to be treated, the condition of the individual patient, the site of delivery, the method of administration and other factors known to practitioners. Examples of techniques and protocols can be found in Remington 's Parmaceutical Sciences. Typically the active agents of the present invention exhibit their effect at a dosage range from about 0.001 mg/kg to about 250 mg/kg, preferably from about 0.05 mg/kg to about 100 mg/kg of the active ingredient, more preferably from a bout 0.1 mg/kg to about 75 mg/kg. A suitable dose can be administered in multiple sub-doses per day. Typically, a dose or sub-dose may contain from about 0.1 mg to about 500 mg of the active ingredient per unit dosage form. A more preferred dosage will contain from about 0.5 mg to about 100 mg of active ingredient per unit dosage form. Dosages are generally initiated at lower levels and increased until desired effects are achieved.

For the treatment of epilepsy, a typical dose of an active agent of Formula I having antagonist activity is in the range of about 1 mg/day to about 4000 mg/day, preferably about 1 mg/day to about 2000 mg/day, usually in 1 to 4 divided dosages, for an average adult human. A unit dosage would contain about 1 mg to about 500 mg of the active ingredient.

For the treatment of pain, a typical dose of an active agent of Formula I having antagonist activity is in the range of about 1 ng/day and 4000 milligrams/day, preferably about 1 ng/day to about 2000 mg/day, more preferably about 1 ng/day to about 1000 mg/day, depending on the mode of delivery. If the route of administration is directly to the central nervous system, the dosage contemplated is between about 1 ng-100 mg per day, preferably between about 100 ng-10 mg per day, most preferably between 1 μg and 100 μg per day. If administered peripherally (e.g., orally, subcutaneously or intravenously, preferably intravenously), the dosage contemplated is somewhat higher, between about 100 ng-4000 mg per day, preferably between about 10 μg-2000 mg per day, most preferably between 100 μg and 1000 mg per day. If the contulakin is administered by continuous infusion (i.e., by pump delivery or bioerodable polymer delivery), then a lower dosage is contemplated than for bolus delivery.

### EXAMPLES

The present invention is described by reference to the following Examples, which are offered by way of illustration and are not intended to limit the invention in any manner. Standard techniques well known in the art or the techniques specifically described below were utilized.

### Example 1

### In vitro Pharmacological Profile of CGX-031-120

The in vitro pharmacological profile of CGX-031-120 was compared to melatonin using radioligand binding assays and assays on effect on intracellular concentrations of cyclic AMP in the assays described by Faust et al. (2000). The results are shown in Tables 1 and 2.

TABLE 1
Radioligand Binding NIH-3T3 Cells vs 2-[¹²⁵I]-Melatonin

| | CGX-031-120 | Melatonin |
|---|---|---|
| K₁mt₁ (human) | 4.37 nM | 0.66 nM |
| K₁MT₂ (human) | 0.17 nM | 0.33 nM |
| MT₂ Selectivity | 26 | 2 |

TABLE 2
Forskolin Stimulated cAMP Release from NIH-3T3 Cells

| | CGX-031-120 | Melatonin |
|---|---|---|
| EC₅₀ (MT₂) | 0.05 nM | 0.15 nM |
| EC₅₀ (mt₁) | 2.2 nM | 0.08 nM |
| MT₂ Selectivity | 44 | 0.5 |

### Example 2

### In vivo Activity of CGX-031-120 in Frings Audiogenic Seizure Susceptible Mice

In vivo anticonvulsant activity of CGX-031-120 and melatonin were analyzed in Frings audiogenic seizure susceptible mice as described by White et al. (1992). CGX-031-120 is found to have anti-seizure activity in this model. The results are shown in Table 3-5.

TABLE 3
Time Effect of CGX-031-120 Against Audiogenic Seizure
Susceptibility of Frings Mice Following i.p. Administration

| | | Time (hrs) | | | | | |
|---|---|---|---|---|---|---|---|
| | Dose | 1/4 | 1/2 | 1 | 2 | 4 | Reference |
| # Prot./# Tested | 100 mg/kg | 1/4 | 2/4 | 3/4 | 4/4 | 4/4 | HA2:175 |
| # Toxic/# Tested | | | | | | | |

TABLE 4
Effect of CGX-031-120 on the Audiogenic Seizure
Susceptibility of Frings Mice Following i.p. Administration

| | Seizure | # Protected/ | | # Toxic/ | |
|---|---|---|---|---|---|
| Dose | Score ± | # Tested | ED₅₀ | # Tested | TD₅₀ |
| (mg/kg) | S.E.M. | (at 2 hr) | (mg/kg) | (at 2 hr) | (mg/kg) |
| 50 | 4.5 ± 0.5 | 1/8 | | | |
| 75 | 4.0 ± 0.65 | 2/8 | | | |
| 87.5 | 2.38 ± 0.78 | 5/8 | 77.0 | | |
| 100 | 1.0 ± 0.0 | 8/8 | (62.3-89.3)* | | |
| 300 | | | | 0/8 | >600 |
| 600 | — | — | | 0/4 | |

*95% confidence interval
Ref: HA2:175, 176, 179, 183, 185

TABLE 5
Effect of CGX-031-120 on the Audiogenic Seizure
Susceptibility of Frings Mice Following i.p. Administration

| | CGX-031-120 | Melatonin |
|---|---|---|
| ED₅₀ (mg/kg) | 77 (62.3-89.3) | 82 (63-101) |
| TD₅₀ (mg/kg) | >900 | 342 (292-381) |
| Protective Index | >12 | 4.2 |

Specifically, the effective dose (ED₅₀) of CGX-031-120 is 77 mg/kg and the toxic dose (TD₅₀) is >900 mg/kg. This compares to an ED₅₀ and TD₅₀ for melatonin of 83 mg/kg and 342 mg/kg, respectively. The protective index (PI; TD₅₀/ED₅₀) for CGX-031-120 is >12, whereas the PI for melatonin is 4. This data illustrates that there is a clear separation of the motor toxicity induced by melatonin and that induced by selective CGX-031-120, i.e., an MT₂ agonist. Similar results are seen for the acetamido analog (CGX-031-122) [ED50=120 mg/kg and TD50=>1000 mg/kg, Protective Index (P.I.) 8.33]

### Example 3

### Comparison of In vivo Activity of CGX-031-120 and Standards in Frings Audiogenic Seizure Susceptible Mice

The anticonvulsant profile of CGX-031-120 and the standards set forth in Table 6 was determined using Frings audiogenic seizure-susceptible mice (25-30 g body weight) obtained from the house colony of the University of Utah. All compounds were administered i.p. Varying doses of the compounds were tested. At the predetermined time of peak anticonvulsant effect, individual mice were placed into a round Plexiglas chamber (diameter, 15 cm; height, 18 cm) pitted with an audio transducer (Model A5-ZC; FET Research & Development, Salt Lake City, Utah) and exposed to a high intensity sound stimulus (110 decibels, 11 KHz) for 25 seconds. Animals not displaying tonic forelimb or hindlimb extension were considered protected. The effect of the test compounds on motor performance was assessed by the rotorod test (Dunham and Miya, 1957). For this procedure, mice were tested for their ability to maintain balance on a rotating (6 rpm) knurled Plexiglas rod (1 inch diameter) for one minute. Mice unable to maintain balance in three successive trials during the test period were considered toxic. The median effective dose (ED₅₀) and the median toxic dose (TD₅₀) was calculated by probit analysis (Finney, 1971). For these studies, the dose of each test substance was varied between the limits of 0 and 100% protection and toxicity. The protective index (PI) is TD₅₀/ED₅₀. The results are shown in Table 6.

TABLE 6
Comparative Anticonvulsant Profiles of CGX-031-120 and Clinically
Used Anti-Seizure Compounds in Frings Audiogenic Mice Following
I.P. Administration

| Test | ED₅₀ | TD₅₀ | |
|---|---|---|---|
| Substance | (mg/kg) | (mg/kg) | P.I. |
| Tegratol ® | 11.2 | 45.4 | 4.1 |
| Klonipin ® | 0.10 | 0.26 | 2.6 |
| Zarontin ® | 328 | 340 | 1.04 |
| Felbatol ® | 10 | 220 | 22 |
| Dilantin ® | 3.9 | 41 | 10.5 |
| Depakote ® | 155 | 398 | 2.6 |
| CGX-031-120 | 77 | >800 | >10 |

### Example 4

### In vivo Activity of CGX-031-120 in CF No. 1 Mice

In vivo anticonvulsant activity of CGX-031-120 is analyzed in CF No. 1 mice as described by White et al. (1995), using the maximal electroshock, subcutaneous pentylenetetrazole (Metrazol) seizure threshold and threshold tonic extension test. CGX-031-120 is found to have anticonvulsant activity in these tests.

### Example 5

### In vivo Phencyclidine-Like Behavioral Effects of CGX-031-120 Following I.C.V. Administration

The in vivo phencyclidine-like behavioral effects of CGX-031-120 is assessed by the elevated platform test as described by Evoniuk et al. (1991). The platform test is a rapid method for evaluating the behavioral effects of phencyclidine-like dissociative anesthetics in mice. At 15 minutes following a administration of CGX-031-120 i.c.v. to mice, no drug-induced falls from the elevated platform are observed. Alternatively, as a control, a 44.5 nmol dose of MK 801 (dizocilpine) elicited 87.5% drug-induced falls from the elevated platform. Thus, CGX-031-120 does not induce phencyclidine-like behavioral effects in mice.

### Example 6

### In Vivo Activity of CGX-031-120 in Pentylenetetrazole-Induced Threshold Seizure Model

The in vivo activity of CGX-031-120 is analyzed using timed intravenous infusion of pentylenetetrazole (White et al., 1995). At time to peak effect, the convulsant solution (0.5% pentylenetetrazole in 0.9% saline containing 10 U.S.P. units/ml heparin sodium) is infused into the tail vein at a constant rate of 0.34 ml/min. The time in seconds from the start of the infusion to the appearance of the first twitch and the onset of clonus is recorded for each drug treated or control animal. The times to each endpoint are converted to mg/kg of pentylenetetrazole for each mouse, and mean and standard error of the mean are calculated. Administration of CGX-031-120 i.c.v. elevates the i.v. pentylenetetrazole seizure threshold, further demonstrating its anti-convulsant activity.

### Example 7

### In Vivo Activity of CGX-031-120 in Maximum Electroshock Seizure Model

The in vivo activity of CGX-031-120 is analyzed using the maximum electroshock (MES) test as described by Syinyard et al. (1952) and Woodbury et al. (1952). In the MES procedure, a tonic seizure is produced in mice (17-26 g) by the delivery of a 50 milliamps current through corneal electrodes for 0.2 sec. Before testing, the animals are allowed food and water ad libitum. Compounds are injected i.p. 30 min before the MES. Reference compounds (such as phenytoin, carbamazepine, phenobarbital, and valproic acid) are tested at the peak of activity and at the dose range reported in the literature. Several mice are used per group. In this test anticonvulsive activity is indicated by the abolition of the hind limb tonic extension. The test compound CGX-031-120 is found to have anti-convulsant activity in the MES test.

### Example 8

### Analgesic Activity of CGX-031-120

Intrathecal (it) drug injections are performed as described by Hylden et al. (1980). CGX-031-120 (2.5 nmol) or water vehicle is administered to CF-1 mice (five mice per group) in a volume of 5 μl. Twenty minutes after injection, the body temperature of each animal is determined. Thirty minutes after injection, each animal is placed on a 55 C. hotplate. The latency to the first response (flinch), a spinally mediated behavioral response, and the first hindlimb lick, a centrally organized motor response to acute pain, are recorded. Mice are removed from the hotplate after 60 seconds if no response is observed. Forty-five minutes after injection, motor function for each mouse is tested by determining the latency to first fall from an accelerating rotarod. The results of these experiments demonstrate that CGX-031-120 has potent analgesic properties

### Example 9

### Analgesic Activity of CGX-031-120

Analgesic activity of CGX-031-120 is also tested in a persistent pain model as follows.

Persistent pain (formalin test). Intrathecal (it) drug injections are performed as described by Hylden and Wilcox (1980). CGX-031-120 or vehicle is administered in a volume of 5 μl. Fifteen minutes after the it injection, the right hindpaw is injected with 20 μl of 5% formalin. Animals are placed in clear plexiglass cylinders backed by mirrors to facilitate observation. Animals are closely observed for 2 minutes per 5 minute period, and the amount of time the animal spends licking the injected paw is recorded in this manner for a total of 45-50 minutes. Results are expressed as licking time in seconds per five minutes. At the end of the experiment, all animals are placed on an accelerating rotorod and the latency to first fall is recorded. CGX-031-120 is found to be active in this model which is predictive of efficacy for treating neuropathic pain.

### Example 10

### Analgesic Activity of CGX-031-120

Analgesic activity of CGX-031-120 is also tested in further pain models as follows.

Acute pain (tail-flick). CGX-031-120 or saline is administered intrathecally (i.t.) according to the method of Hylden and Wilcox (1980) in a constant volume of 5 μl. Mice are gently wrapped in a towel with the tail exposed. At various time-points following the i.t. injection, the tail is dipped in a water bath maintained at 54 C. and the time to a vigorous tail withdrawal is recorded. If there is no withdrawal by 8 seconds, the tail is removed to avoid tissue damage.

Neuropathic pain. The partial sciatic nerve ligation model is used to assess the efficacy of CGX-031-120 in neuropathic pain. Nerve injury is produced according to the methods of Malmberg and Basbaum (1998). Animals are anesthetized with a ketamine/xylazine solution, the sciatic nerve is exposed and tightly ligated with 8-0 silk suture around ⅓ to ½ of the nerve. In sham-operated mice the nerve is exposed, but not ligated. Animals are allowed to recover for at least 1 week before testing is performed. On the testing day, mice are placed in plexiglass cylinders on a wire mesh frame and allowed to habituate for at least 60 minutes. Mechanical allodynia is assessed with calibrated von Frey filaments using the up-down method as described by Chaplan et al. (1994), and the 50% withdrawal threshold is calculated. Animals that did not respond to any of the filaments in the series are assigned a maximal value of 3.6 grams, which is the filament that typically lifted the hindlimb without bending, and corresponds to approximately 1/10 the animal's body weight.

The data obtained demonstrate that CGX-031-120 has potent analgesic properties in three commonly used models of pain: acute, persistent/inflammatory and neuropathic pain models. CGX-031-120 administered intrathecally reduces the response latency in the tail flick model of acute pain. CGX-031-120 also shows analgesic activity in a model of neuropathic pain.

### Example 11

### Analgesic Activity in Persistent Pain Model

Melatonin and three analogs (CGX-031133, CGX-031139 and CGX-MTAG) were compared in the formalin model of persistent pain. In this model, two distinct phases of nociceptive activity (paw licking) are observed. Phase one lasts approximately 10 minutes, and is thought to be caused by the direct action of formalin in activating nociceptive c-fibers, and is a model of acute, chemically induced pain. The second phase is caused by the release of inflammatory factors from tissue damage caused by formalin injection. This phase lasts for 30 to 40 minutes, and is a model of persistent inflammatory pain. The second phase is also predictive of analgesic drugs that will show efficacy in models of chronic and neurophathic pain.

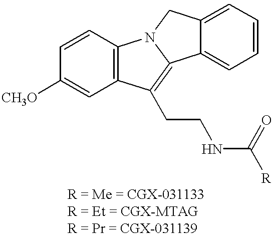

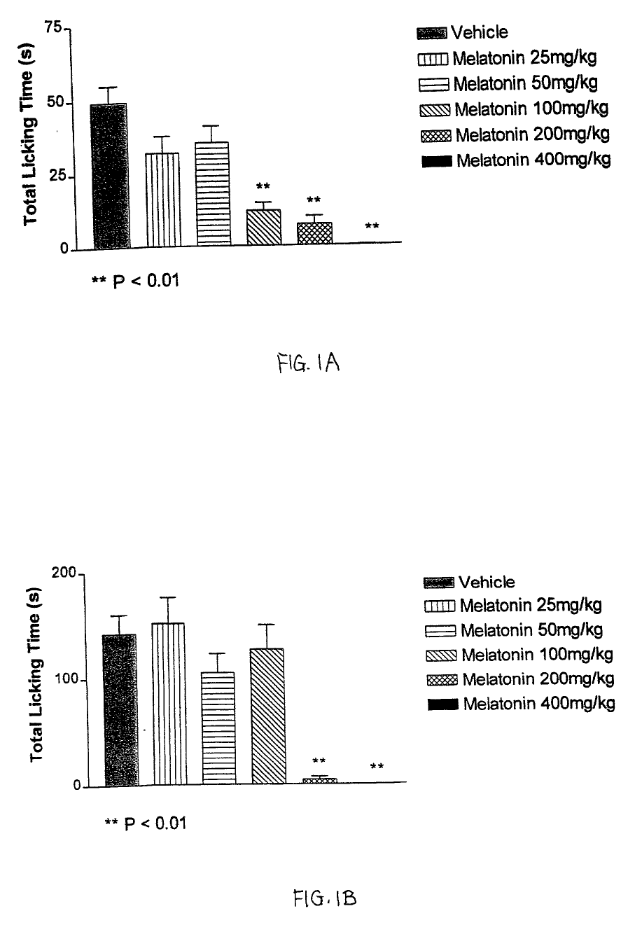

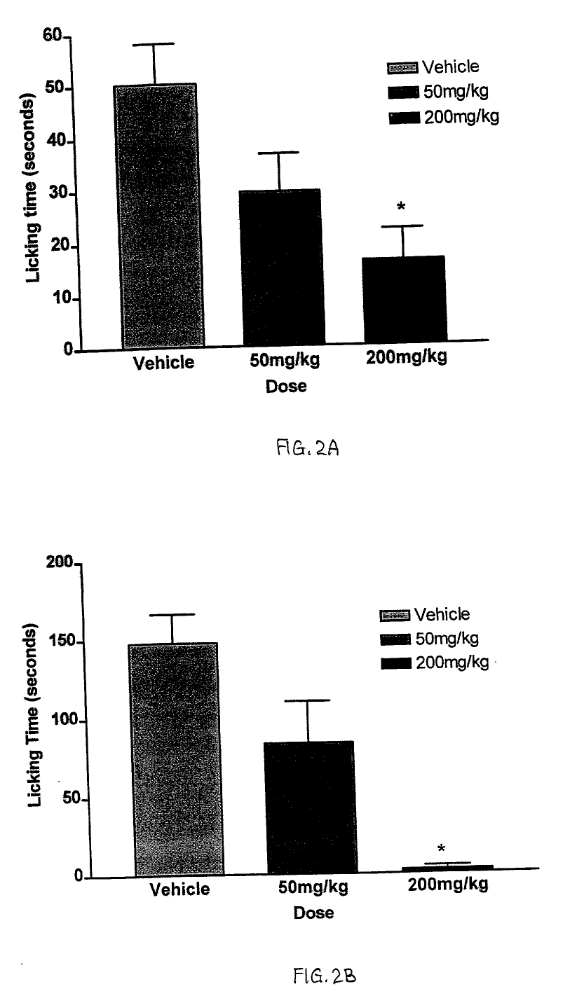

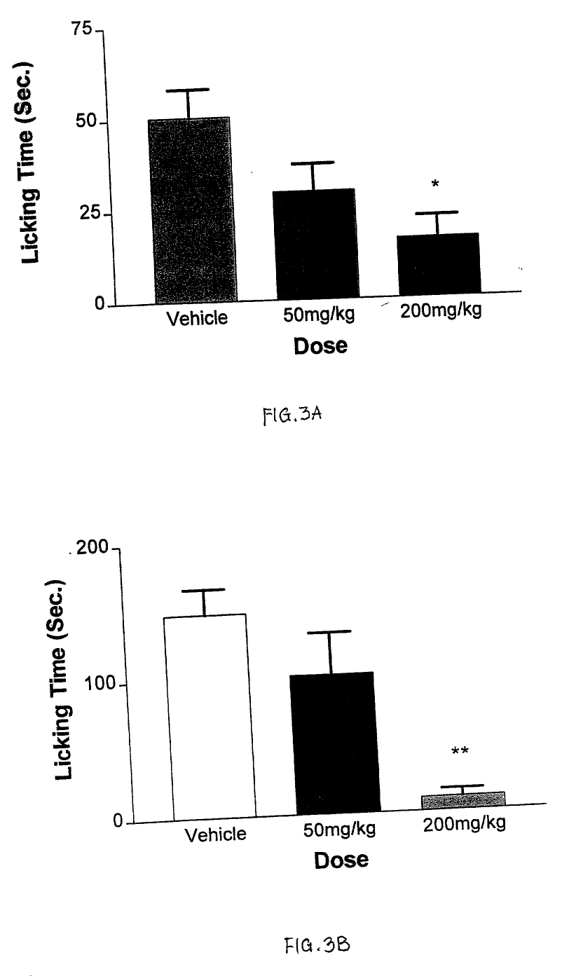

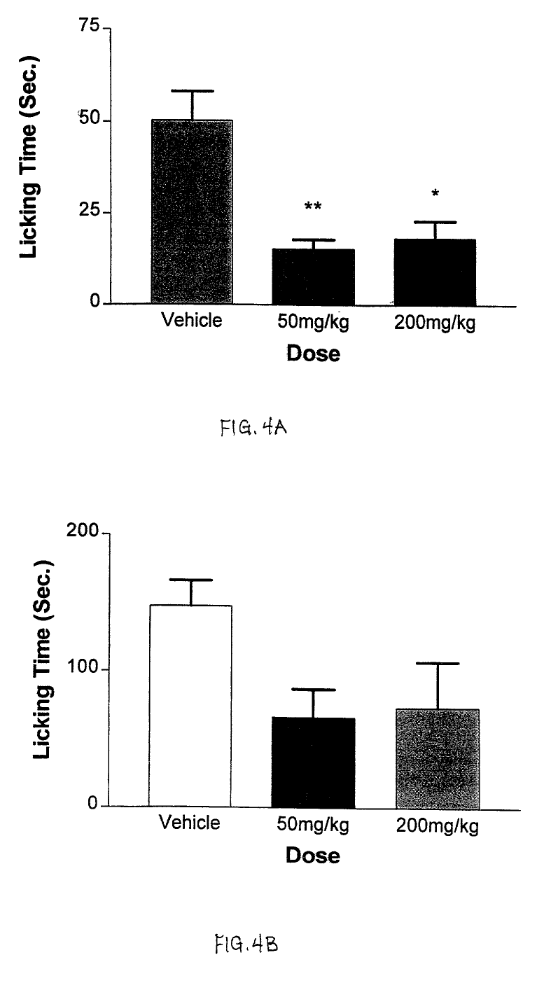

A full dose response (5 doses) was generated fro melatonin in the formalin test. FIG. 1 shows that melatonin dose-dependently reduced the licking time following formalin injection I both phases of this test, reaching significance at 100 mg/kg and above in phase one, and 200 mg/kg and above in phase two. All three analogs showed significantly reduced licking times in phase one at 200 mg/kg (FIGS. 2A, 3A and 4A) and CGX-MTAG showed a significant reduction at 50 mg/kg (FIG. 4A). In phase two, both CGX-031133 (FIG. 2B) and CGX-031139 (FIG. 3B) completely blocked licking behavior at 200 mg/kg, while CGX-MTAG (FIG. 4B) had no significant effect at the doses tested.

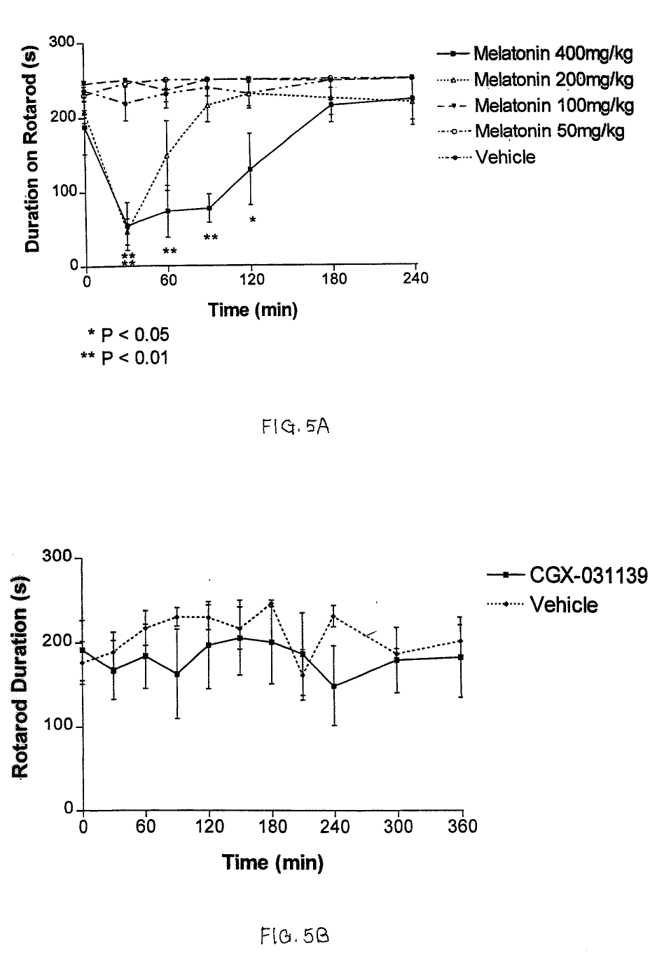

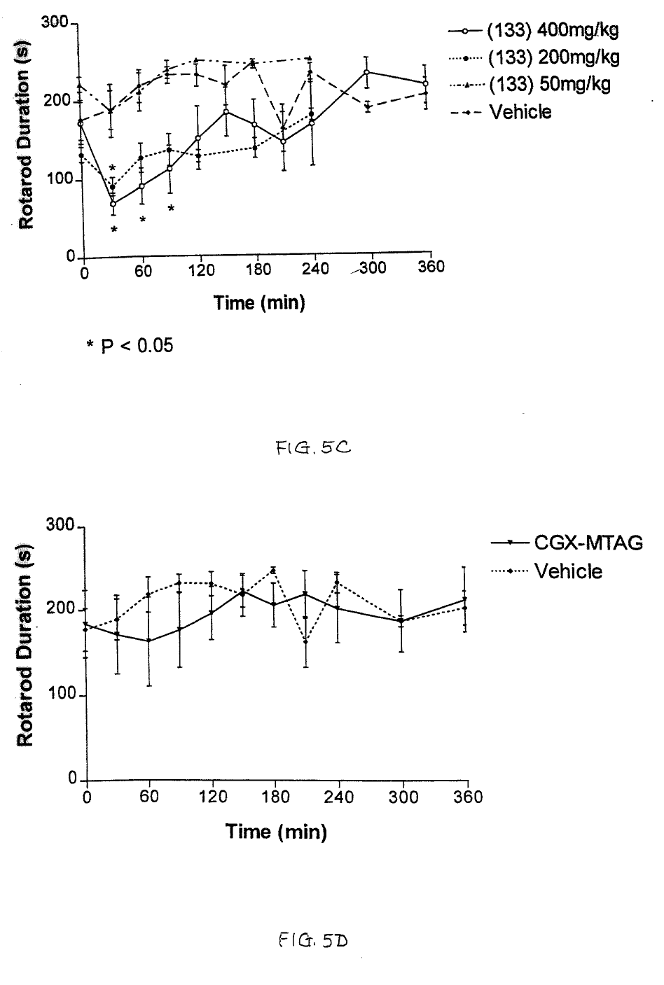

The motor impairing effects of these drugs were compared by measuring the latency to fall from an accelerating rotorod. Melatonin showed short-lasting but significant motor toxicity at 200 mg/kg and long lasting impairment at 400 mg/kg (FIG. 5A). Neither CGX-031139 (FIG. 5B) nor CGX-MTAG (FIG. 5D) showed motor toxicity at 400 mg/kg. Similar to melatonin, CGX-031133 (FIG. 5C) showed significant motor impairment at 200 mg/kg and above.

It will be appreciated that the methods and compositions of the instant invention can be incorporated in the form of a variety of embodiments, only a few of which are disclosed herein. It will be apparent to the artisan that other embodiments exist and do not depart from the spirit of the invention. Thus, the described embodiments are illustrative and should not be construed as restrictive.

### BIBLIOGRAPHY

Arendt et al. (1986). Br. Med. J. 292:1170.

What is claimed is:

1. A method for inducing analgesia in a mammal comprising administering to a mammal in need thereof a therapeutically effective amount of a compound of Formula I having melatonin agonist activity: 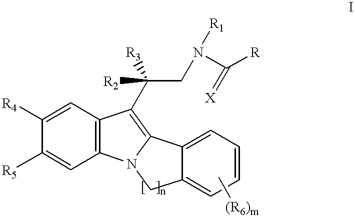
wherein
R is H, a C₁₋₆ alkyl, CF₃, C₂F₅, C₃₋₆ cycloalkyl, —(CH₂)ₚ—C₃₋₆ cycloalkyl, C₂₋₆ alkenyl, C₂₋₆ alkynyl or heterocycle;
R₁ is H or C₁₋₃ alkyl; or
R and R₁ together with the atoms to which they are attached form a heterocyclic ring of 5-7 atoms;
R₂ and R₃ are independently H or C₁₋₃ alkyl; or
R₂ and R₃ together with the atom to which they are attached form a C₃₋₆ cycloalkyl;
R₄ is a H, OR⁷ or SR⁷;
R₅ is H, C₁₋₅ alkyl, phenyl, halogen (preferably F or Cl); or
when R₅ is a C₁₋₅ alkyl, then R₅ may also be linked to R₄ by an O or an S;
R₆ is H, halogen (preferably F or Cl), C₁₋₄ alkyl, C₁₋₄ alkoxy, C₁₋₄ thioalkyl, phenyl or heterocycle;
R₇ is H, C₁₋₆ alkyl or —(CH₂)ₚ—C₃₋₆ cycloalkyl;
X is O, S or NH;
m is 0, 1 or 2
n is 0, 1, 2 or 3; and
p is 0, 1, 2, 3 or4.

2. The method of claim 1, wherein one or more of said alkyls is substituted by halogen atom (e.g., fluorine, chlorine, bromine, iodine), a nitro group, a cyano group, a hydroxy group, an amino group, a carboxy group, a C₁₋₃ alkoxy, a halogenated C₁₋₃ alkyl group, a mono- or di-C₁₋₃ alkylamino group, a C₁₋₃ alkylcarbonyl group, a C₁₋₃ alkoxycarbonyl group, a carbamoyl group, and a mono- or di-C₁₋₃ alkylcarbamoyl group.

3. The method of claim 1, wherein the dosage of the active agent administered is between 1 nanogram/day and 4000 milligrams/day.

4. The method of claim 1, wherein the active agent is administered using a delivery system selected from the group consisting of pump delivery, bioerodable polymer delivery, microencapsulated cell delivery, oral, injection, macroencapsulated cell delivery and patch delivery.

5. The method of claim 4, wherein administration is into the intrathecal space.

6. The method of claim 4, wherein administration is into the ventricular space.

7. The method of claim 4, wherein administration is oral.

8. The method of claim 4, wherein administration is intraparental injection.

9 A method for eliciting an anticonvulsive effect in a mammal comprising administering to a mammal in need thereof a therapeutically effective amount of a compound of Formula I having melatonin agonist activity: 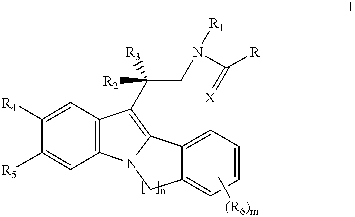
wherein
R is H, a C₁₋₆ alkyl, CF₃, C₂F₅, C₃₋₆ cycloalkyl, —(CH₂)ₚ—C₃₋₆ cycloalkyl, C₂₋₆ alkynyl or heterocycle;
R₁ is H or C₁₋₃ alkyl; or
R and R₁ together with the atoms to which they are attached form a heterocyclic ring of 5-7 atoms;
R₂ and R₃ are independently H or C₁₋₃ alkyl; or
R₂ and R₃ together with the atom to which they are attached form a C₃₋₆ cycloalkyl;
R₄ is a H, OR⁷ or SR⁷;
R₅ is H, C₁₋₅ alkyl, phenyl, halogen (preferably F or Cl); or
when R₅ is a C₁₋₅ alkyl, then R₅ may also be linked to R₄ by an O or an S;
R₆ is H, halogen (preferably F or Cl), C₁₋₄ alkyl, C₁₋₄ alkoxy, C₁₋₄ thioalkyl, phenyl or heterocycle;
R₇ is H, C₁₋₆ alkyl or —(CH₂)ₚ—C₃₋₆ cycloalkyl;
X is O, S or NH;
m is 0, 1 or 2
n is 0, 1, 2 or 3; and
p is 0, 1, 2, 3 or 4.

10. The method of claim 9, wherein one or more of said alkyls is substituted by halogen atom (e.g., fluorine, chlorine, bromine, iodine), a nitro group, a cyano group, a hydroxy group, an amino group, a carboxy group, a C₁₋₃ alkoxy, a halogenated C₁₋₃ alkyl group, a mono- or C₁₋₃ alkylamino group, a C₁₋₃ alkylcarbonyl group, a C₁₋₃ alkoxycarbonyl group, a carbamoyl group, and a mono- or di-C₁₋₃ alkylcarbamoyl group.

11. The method of claim 9, wherein the dosage of the active agent administered is between 1 ng/day and 4000 milligrams/day.

12. The method of claim 9, wherein the active agent is administered using a delivery system selected from the group consisting of pump delivery, bioerodable polymer delivery, microencapsulated cell delivery, oral, injection, macroencapsulated cell delivery and patch delivery.

13. The method of claim 12, wherein administration is into the intrathecal space.

14. The method of claim 12, wherein administration is into the ventricular space.

15. The method of claim 12, wherein the administration is oral.

16. The method of claim 12, wherein the administration is intraparental injection.

17. A method for treating a disorder arising from overproduction of melatonin in a mammal comprising administering to a mammal in need thereof a therapeutically effective amount of a compound of Formula I having melatonin antagonist activity: 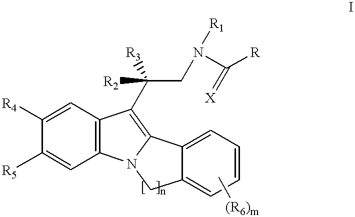
wherein
R is H, a C₁₋₆ alkyl, CF₃, C₂F₅, C₃₋₆ cycloalkyl, —(CH₂)ₚ—C₃₋₆ cycloalkyl, C₂₋₆ alkenyl, C₂₋₆ alkynyl or heterocycle;
R₁ is H or C₁₋₃ alkyl; or
R and R₁ together with the atoms to which they are attached form a heterocyclic ring of 5-7 atoms;
R₂ and R₃ are independently H or C₁₋₃ alkyl; or
R₂ and R₃ together with the atom to which they are attached form a C₃₋₆ cycloalkyl;
R₄ is a H, OR⁷ or SR⁷;
R₅ is H, C₁₋₅ alkyl, phenyl, halogen (preferably F or Cl); or
when R₅ is a C₁₋₅ alkyl, then R₅ may also be linked to R₄ by an O or an S;
R₆ is H, halogen (preferably F or Cl), C₁₋₄ alkyl, C₁₋₄ alkoxy, C₁₋₄ thioalkyl, phenyl or heterocycle;
R₇ is H, C₁₋₆ alkyl or —(CH₂)ₚ—C₃₋₆ cycloalkyl;
X is O, S or NH;
m is 0, 1 or 2
n is 0, 1, 2 or 3; and
p is 0, 1, 2, 3 or 4.

18. The method of claim 17, wherein one or more of said alkyls is substituted by halogen atom (e.g., fluorine, chlorine, bromine, iodine), a nitro group, a cyano group, a hydroxy group, an amino group, a carboxy group, a C₁₋₃ alkoxy, a halogenated C₁₋₃ alkyl group, a mono- or di-C₁₋₃ alkylamino group, a C₁₋₃ alkylcarbonyl group, a C₁₋₃ alkoxycarbonyl group, a carbamoyl group, and a mono- or di-C₁₋₃ alkylcarbamoyl group.

19. The method of claim 17, wherein the dosage of the active agent administered is between 1 nanogram/day and 4000 milligrams/day.

20. The method of claim 17, wherein the active agent is administered using a delivery system selected from the group consisting of pump delivery, bioerodable polymer delivery, microencapsulated cell delivery, oral, injection, macroencapsulated cell delivery and patch delivery.

21. The method of claim 20, wherein administration is into the intrathecal space.

22. The method of claim 20, wherein administration is into the ventricular space.

23. The method of claim 20, wherein administration is oral admistration.

24. The method of claim 20, wherein administration is intrparental injection.

25. The method of claim 17, wherein said disorder is seasonal affective disorder or circadian rhythm disorder.

26. A method for treating Parkinson's disease in a mammal comprising administering to a mammal in need thereof a therapeutically effective amount of a compound of Formula I having melatonin antagonist activity: 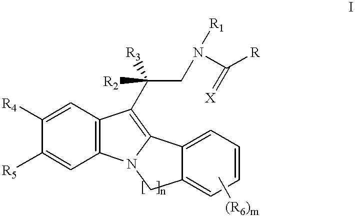
wherein
R is H, a C₁₋₆ alkyl, CF₃, C₂F₅, C₃₋₆ cycloalkyl, —(CH₂)ₚ—C₃₋₆ cycloalkyl, C₂₋₆ alkenyl, C₂₋₆ alkynyl or heterocycle;
R₁ is H or C₁₋₃ alkyl; or
R and R₁ together with the atoms to which they are attached form a heterocyclic ring of 5-7 atoms;
R₂ and R₃ are independently H or C₁₋₃ alkyl; or
R₂ and R₃ together with the atom to which they are attached form a C₃₋₆ cycloalkyl;
R₄ is a H, OR⁷ or SR⁷;
R₅ is H, C₁₋₅ alkyl, phenyl, halogen (preferably F or Cl); or
when R₅ is a C₁₋₅ alkyl, then R₅ may also be linked to R₄ by an O or an S;
R₆ is H, halogen (preferably F or Cl), C₁₋₄ alkyl, C₁₋₄ alkoxy, C₁₋₄ thioalkyl, phenyl or heterocycle;
R₇ is H, C₁₋₆ alkyl or —(CH₂)ₚ—C₃₋₆ cycloalkyl;
X is O, S or NH;
m is 0, 1 or 2
n is 0, 1, 2 or 3; and
p is 0, 1, 2, 3 or 4.

27. The method of claim 26, wherein one or more of said alkyls is substituted by halogen atom (e.g., fluorine, chlorine, bromine, iodine), a nitro group, a cyano group, a hydroxy group, an amino group, a carboxy group, a C₁₋₃ alkoxy, a halogenated C₁₋₃ alkyl group, a mono- or di-C₁₋₃ alkylamino group, a C₁₋₃ alkylcarbonyl group, a C₁₋₃ alkoxycarbonyl group, a carbamoyl group, and a mono- or di-C₁₋₃ alkylcarbamoyl group.

28. The method of claim 26, wherein the dosage of the active agent administered is between 1 nanogram/day and 4000 milligrams/day.

29. The method of claim 26, wherein the active agent is administered using a delivery system selected from the group consisting of pump delivery, bioerodable polymer delivery, microencapsulated cell delivery, oral, injection, macroencapsulated cell delivery and patch delivery.

30. The method of claim 29, wherein administration is into the intrathecal space.

31. The method of claim 29, wherein administration is into the ventricular space.

32. The method of claim 29, wherein administration is oral admistration.

33. The method of claim 29, wherein administration is intrparental injection.

34. The method of claim 26, wherein said disorder is seasonal affective disorder or circadian rhythm disorder.
